(19) United States
(12) Patent Application Publication
(10) Pub. No.: US 2014/0163352 A1
(43) Pub. Date: Jun. 12, 2014

(54) Portable Tonometer

(71) Applicants: Francis Y. Falck, JR., Stonington, CT (US); Robert W. Falck, Pawcatuck, CT (US)

(72) Inventors: Francis Y. Falck, JR., Stonington, CT (US); Robert W. Falck, Pawcatuck, CT (US)

(73) Assignee: FALCK MEDICAL, INC., Mystic, CT (US)

(21) Appl. No.: 13/932,113

(22) Filed: Jul. 1, 2013

Related U.S. Application Data
(60) Provisional application No. 61/666,983, filed on Jul. 2, 2012.

(51) Int. Cl. A61B 3/16; A61B 3/00

(52) U.S. Cl. CPC A61B 3/16; A61B 3/0008; A61B 3/0041; A61B 3/0025
USPC 600/399

(57) ABSTRACT

An applanation tonometer uses an active pixel sensor array to accurately detect total illumination in a region surrounding an applanated area so that a microprocessor can determine the applanated area based on an inversion of the illumination received by the sensor.

### TECHNICAL FIELD

### Tonometry

### RELATED APPLICATIONS

This application claims benefit under 35 USC §119(e) of subject matter disclosed in provisional application No. 61/666,983, filed 2 Jul. 2012 entitled “Portable Tonometer”.

### BACKGROUND

Our previous tonometer patents and applications (U.S. Pat. No. 5,070,875; 6,179,779; 6,471,647; 6,736,778; 7,153,267; 7,473,231; 7,479,109; and Publications No. 2009/0103047; and 2011/0087086) produce accurate results, especially when mounted on slit lamp microscopes. This allows evenly applied force of an applanator against the cornea of an eye while measuring the size of an applanated area.

Such accurate results are more difficult to reach when using a handheld portable device. Humanly applied force pressing an applanator against a cornea cannot be applied as evenly as a mechanism on a slit lamp microscope allows. It is also challenging to provide all the necessary structures in a workable form in a compact handheld portable device. Some prior art devices, for example, suggest equipment that is far too cumbersome to be deployed in a hand operated device. Cost is also a factor, and if a handheld device is to be widely available, it must have an affordable price.

### SUMMARY

We have discovered a combination of structures that meet many of the goals for portable tonometers. These goals include ease of operation, accuracy of results, durability of the device, reproducibility of the results, and the ability to download intraocular pressure (TOP) data. The goals also include making all the measurements suggested in our previous patents, including IOP, ocular pulse amplitude (OPA), diastolic and systolic variations in the cardiac cycle as evidenced in eye examinations, and measuring the resistance of the trabecular mesh work, and the amount of force required to observe pulsation of the central retinal artery. Such measurements are explained in more detail in our previous patents and published applications listed above, and the goal of the portable tonometer is to make all these measurements with different elements performing the same tonometry functions. Low cost is also desirable for a portable device so that affordable tonometers can be made widely available. This presents the challenge of embodying desirable capabilities into portable tonometers that are both inexpensive and highly accurate.

### DRAWINGS

FIGS. 1 and 2 are respective side and front views of a portable tonometer embodiment.

FIG. 3 is a rear view of the tonometer of FIGS. 1 and 2 showing an operator-visible display.

FIG. 4 is a partially schematic elevational view of operating components of a portable tonometer embodiment.

FIG. 5 is a partially schematic view of an applanator tab and bend sensor to ensure that a fresh applanator is used with each examination of a new pair of eyes.

FIG. 6 is a perspective view of another preferred embodiment of an applanator.

FIG. 7 is a front view of the applanator of FIG. 6.

FIG. 8 is a perspective view of a portable tonometer using active pixel sensors.

FIG. 9 is a perspective view from another angle of the embodiment of FIG. 8.

FIG. 10 is a fragment of the embodiment of FIG. 9 showing schematically how an applanator is removed and installed.

FIG. 11 is a schematic view of an applanator support arm and driving system for an active pixel sensor tonometer such as shown in FIGS. 8-10.

FIG. 12 is a graphic diagram of voltage output and reset signals for an active pixel.

### DETAILED DESCRIPTION

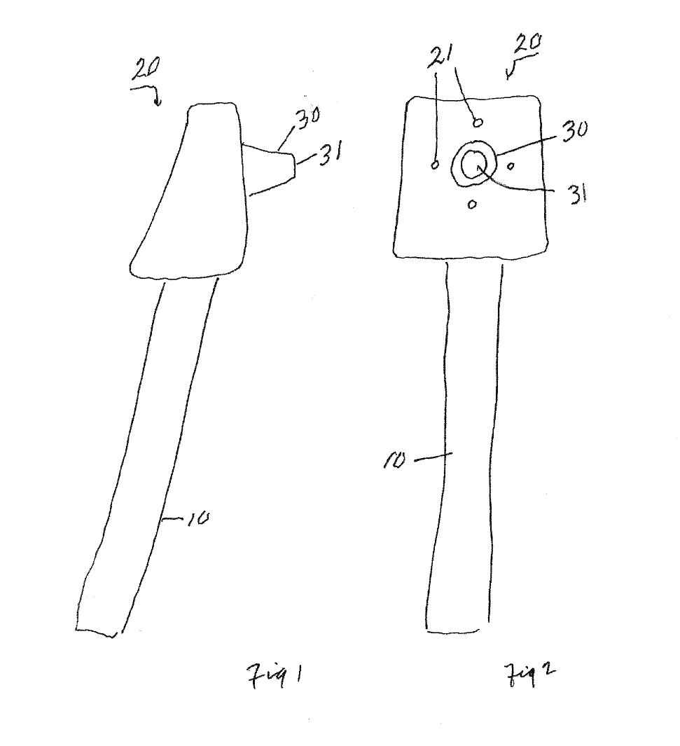

The portable tonometer embodiment illustrated in FIGS. 1-3 includes a handle 10, preferably containing batteries (not shown), and an operating head 20. An operator gripping handle 10 will aim applanator 30 at an eye to be examined and will move the applanator 30 gently toward the eye. A display 40 facing the operator will make the applanator approach to the eye visible so that the operator can center the applanator on the cornea of the eye. The instrument will notice and record initial contact of surface 31 with the eye, which results in a slight and variable applanation of the eye. This will produce a dark spot 41 in display 40, which can indicate to an operator that applanator 30 is centered on the eye.

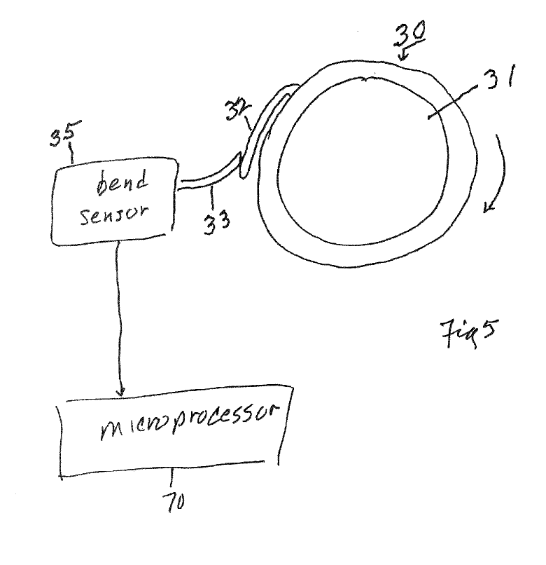

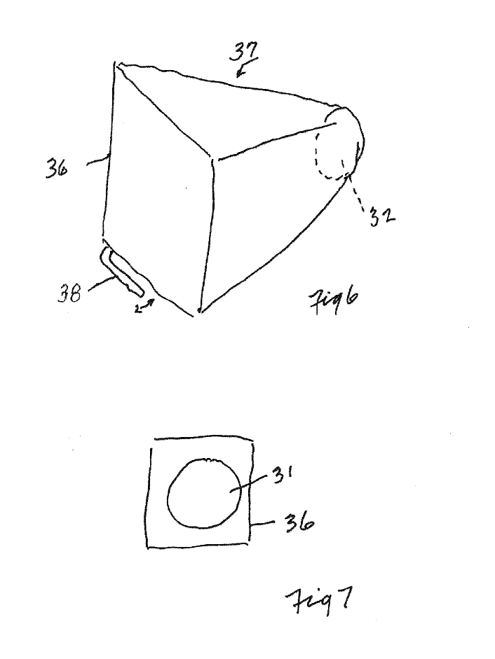

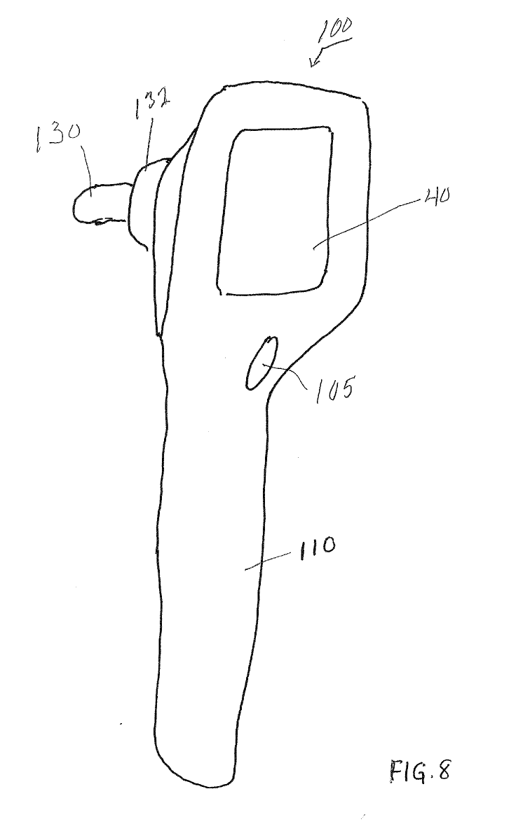

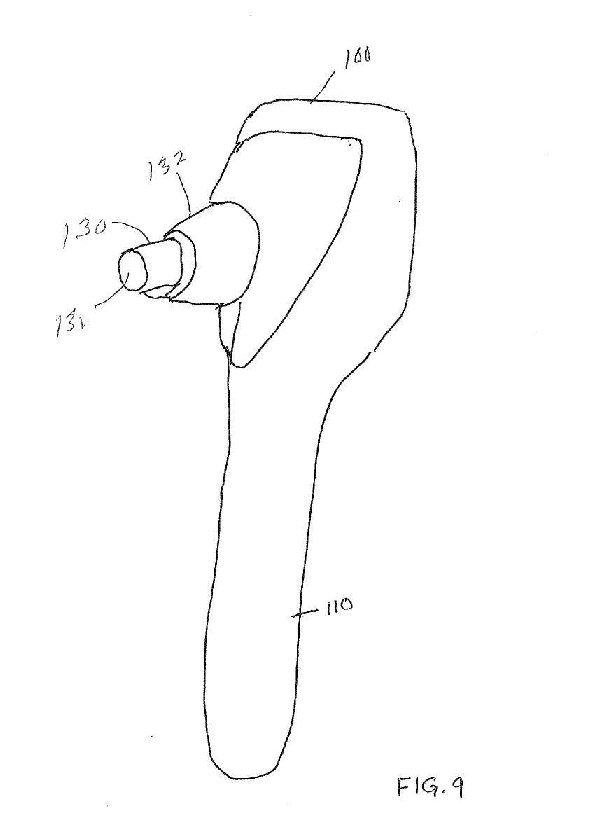

Applanator 30, as shown in FIGS. 1, 2, 4, and 5 is preferably molded of a resin material. Applanating surface 31 is preferably flat and sized to applanate an effective area of a cornea. The inside surface 32 of applanator 30 is preferably parallel with applanating surface 31. In the illustrated embodiment applanator 30 has approximately the shape of a thimble. As shown in FIGS. 6 and 7, the thimble shape can be modified to have a circular applanating surface 31 and a square opening delivering radiation to a detector 50. The applanator shape 30 then becomes a modified trapezoid with a square base and a circular applanator face 31.

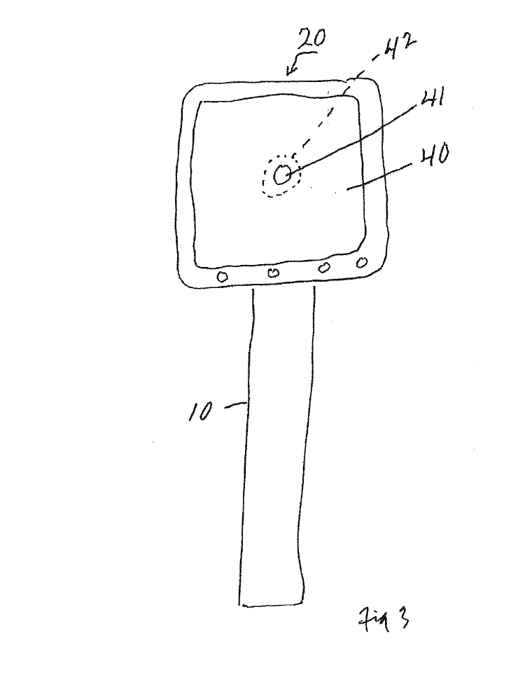

Applanator 30 is structured to be inserted into an operating position in head 20 as shown in FIGS. 1 and 2. After insertion into head 20, applanator 30 is preferably rotated about a quarter turn, as shown by the arrow in FIG. 5. During the partial rotation, a flexible tab 32, preferably formed on an open end of applanator 30, rotates against the flexible arm 33 of bend sensor 35. A limit on the rotation can be set by a detent so that bend sensor 35 detects the fully inserted presence of applanator 30. This can ensure that a fresh applanator 30 is used for each eye examination. Bend sensor 35 can distinguish between a previously used applanator and an unused applanator by the flexibility of tab 32. Bend sensor 35 can also detect when a previously used applanator is removed from an applanator support arm. This prevents anyone from using the same applanator successively on different patients, which can possibly transmit pathogens from one eye to another.

Alternative applanator 37 is preferably inserted directly into head 20 without requiring a partial rotation. Flexible tab 38 is bent upon insertion of applanator 37 to cooperate with a bend sensor detecting placement of a fresh applanator.

The thimble shape of applanator 30 or the modified trapezoidal shape of applanator 37 each have advantages in keeping molding costs low so that applanators can be disposed of after each examination of a pair of eyes. Microprocessor 70 tends to this by not allowing further examinations with a previously used applanator 30. For this purpose, bend sensor 35 communicates to microprocessor 70 the flexible nature of tabs 32 or 38 on respective applanators 30 and 37. Microprocessor 70 can then distinguish between fresh and previously used applanators so as to require replacement of a used applanator before beginning examination of a new pair of eyes.

The hollow interior of applanators 30 or 37 advantageously allows light transmitting through surfaces 31 and 32 to be sensed directly by active pixel sensor 50. Surfaces 31 and 32 are preferably optically flat so as to transmit light from an eye clearly through an applanator to active pixel sensor array 50. Minimizing the number and extent of optical surfaces helps keep the cost of applanators low enough so that they can affordably be disposable.

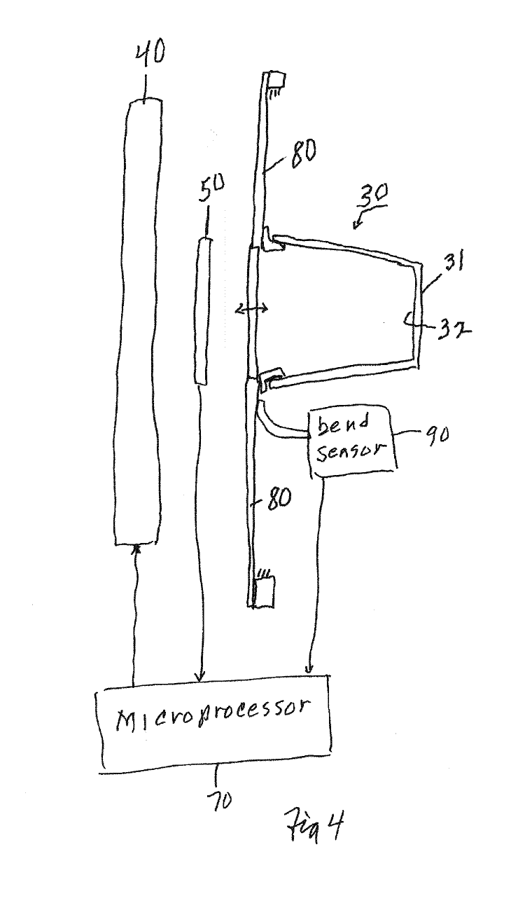

A light source 21 for portable tonometer 20 is shown in FIG. 2 as an array of LEDs arranged in a ring around applanator 30. Four of these lights 21 are illustrated in FIG. 2, and fewer or more small light sources 21 can be used. The light sources used can emit visible light or non visible light such as infrared. Whatever light sources are used must be compatible with active pixel sensor array 50. The preferred result is a ring of light around applanators 30 or 37 illuminating the eye that the applanator will contact. This provides adequate light so that active pixel sensor array 50 can sense the light transmitted through applanator surfaces 31 and 32, and passing through the hollow interior of applanators 30 or 37 to be incident on active pixel array 50. Where applanator surface 31 contacts and applanates a cornea no light will be transmitted through applanators 30 or 37. The result, as illustrated in FIG. 3 is a small dark spot 41 that occurs when applanators 30 or 37 initially contact a cornea. Further force of applanators 30 or 37 against a cornea can flatten or applanate a larger area 42, as shown by a dotted circle in FIG. 3. This results in an inverse relationship between the size of the applanated area and the total illumination occurring around the applanated area.

The illumination of the eye in a region around an applanator helps an operator guide the applanator to the center of a cornea. Display 40 displays an image of the light incident on active pixel sensor 50. This gives an operator a working view of the applanation process.

One way of mounting applanators 30 or 37 is on a flexible diaphragm 80 that has an opening 81 allowing light passing through to reach active pixel sensor array 50. Bend sensor 90 detects movement of diaphragm 80 as applanators 30 or 37 are pressed against a cornea. Bend sensor 90 thus measures directly the force resistance of the cornea. At the same time, active pixel array 50 measures the size of the corneal area that is applanated as surface 31 presses against it. This results in direct measurement of two important variables involved in tonometry. Movement of diaphragm 80 directly measures the force applied to the eye by applanating surface 31. At the same time, active pixel sensor 50 measures the size of the area applanated. These two determinations—the force applied to the eye, the applanation size resulting from such force allows measuring a full set of parameters of an eye.

A hand of an operator of the device applies the force that presses surface 31 against the cornea, and the resistance of the eye to this force is measured directly by bend sensor 90. Differences in force applied by applanator surface 31 also applanate difference sizes of flattened areas of a cornea, which active pixel sensor 50 senses directly. This enables a portable active pixel sensor embodiment to measure directly both eye resistance to applanation and a corneal area that is applanated. This allows IOP and other parameters to be derived directly from the two measurements.

As a measurement proceeds, applanator surface 31 initially contacts a cornea to result in a small area of applanation caused by tears engaging applanating surface 31. This initial contact can be noticed by active pixel sensor array 50 quicker than a human hand can increase the force on the eye. Then the instrument can use the initial contact area as one end of a force range that is increased by hand force. When the hand force reaches a predetermined value beyond the initial contact value, the instrument can then calculate IOP. The human hand may continue the force beyond what is needed for this, but the instrument can help prevent any force damage to the eye by indicating with a light or sound that the examination is completed.

Microprocessor 70 tends to the IOP and other calculations from inputs by active pixel sensor array 50 and bend sensor 90. Microprocessor 70 also operates display 40 to help guide an operator to an accurate registration of the applanator with the eye to ensure a successful examination. The calculations accomplished by microprocessor 70 can be delivered to a computer or other devices dealing with data. For this purpose we prefer wireless connectivity, whether infrared (IR) or radio frequency (RF). Along with data results, user commands can be exchanged with a wireless device. The calculation results are preferably also made visible in display 40. Microprocessor 70 also receives inputs from bend sensor 35, which is arranged to detect the flexibility of applanator tabs 32 or 38 to assure that a new applanator is used for each examination of a new pair of eyes.

The combination of the elements as described above provides two reliable signals of force and area that are used to calculate an accurate IOP. The components are also made simple, lightweight, small, and inexpensive so that a portable instrument can operate reliably and produce accurate results for a small investment. Active pixel sensor array 50 is especially preferred for its low cost ability to receive light from applanator window 31. Display 40 preferably has the ability to image light detected by active pixel sensor 50 and present this image in a viewable and enlarged form that essentially magnifies the applanation function for the convenience of an operator. These features will encourage use of tonometers in the offices and clinics of doctors, veterinarians, optometrists, and others.

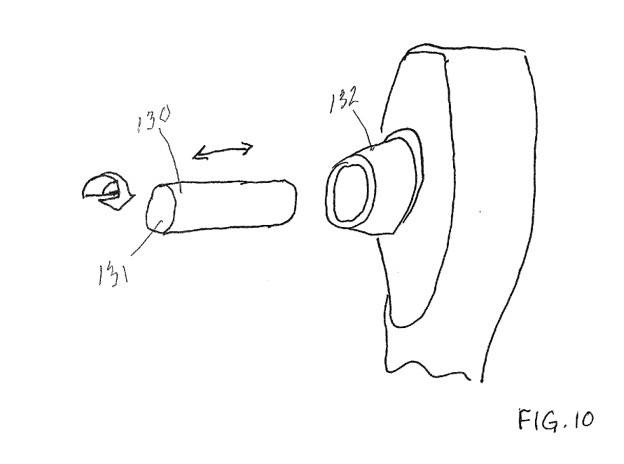

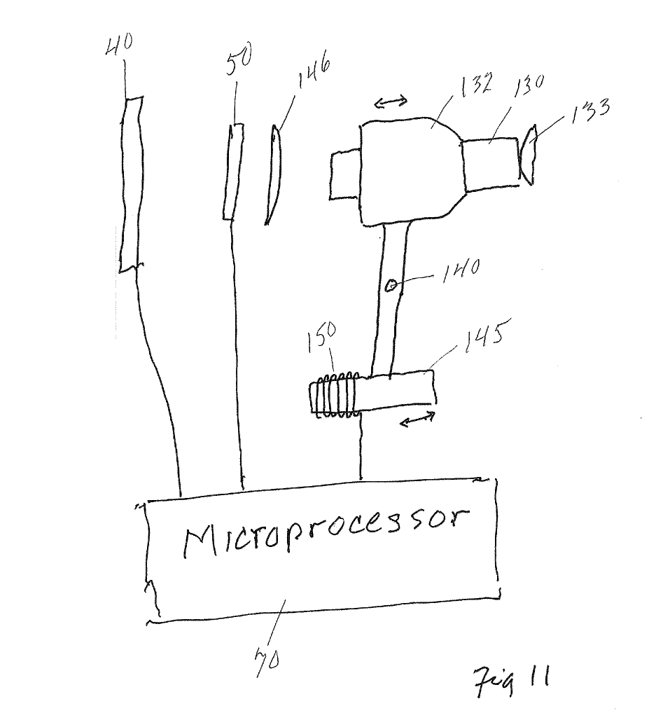

FIGS. 8-11 show another embodiment 100 having a different exterior configuration and a counterbalanced applanator arm operated by a solenoid while otherwise using the above described elements and methods. Embodiment 100 has an applanator 130 held in an applanator support arm 132. Embodiment 100 also has a handle 110 and an on/off switch 105. FIG. 10 shows applanator 130 removed from applanator support arm 132. A double headed arrow shows how applanator 130 can be moved in and out of applanator support arm 132, and a rotational arrow shows how applanator 130 can be rotated within support arm 132 to indicate that applanator 30 is fresh and ready for examination of a pair of eyes. Applanator 130 has a cornea contacting window 131 that operates as described above for surface 31, which applanates a region of a cornea while transmitting light passing through applanator window 131 in a region around the area applanated.

For all the illustrated embodiments, an active pixel sensor array 50 is used for detecting illumination in a region around an applanated area. Each pixel in an active pixel array includes an amplifier, as is generally known, to contribute to the reliability of the active pixels to detect the amount of the light transmitted through an applanator so that a microprocessor 70 can determine from the received light the amount of an area applanated. Since the area applanated is an area that is not illuminated, the determination made by a microprocessor 70 is an inversion of the light received. The more light that is received, the smaller is the applanated area and the less light received the larger the applanated area.

The pixels of an active pixel array respond in an on/off fashion to illumination exceeding a threshold value. This, together with the amplifier included with each pixel, makes the active pixel sensor accurate and reliable in determining the amount of the light transmitted and thus establishing the accuracy of any measurement based on the received illumination. Active pixel sensors also contribute to tonometer accuracy by not requiring any external amplification; by not experiencing any blooming effect from being overloaded with transmitted illumination; and for not experiencing any roll-off around the periphery of the detected light. Digital cameras in other configurations suffer from these problems and are also designed to produce an image, rather than to sense total illumination. This makes them far less desirable than active pixel sensors suitable for accurate tonometry.

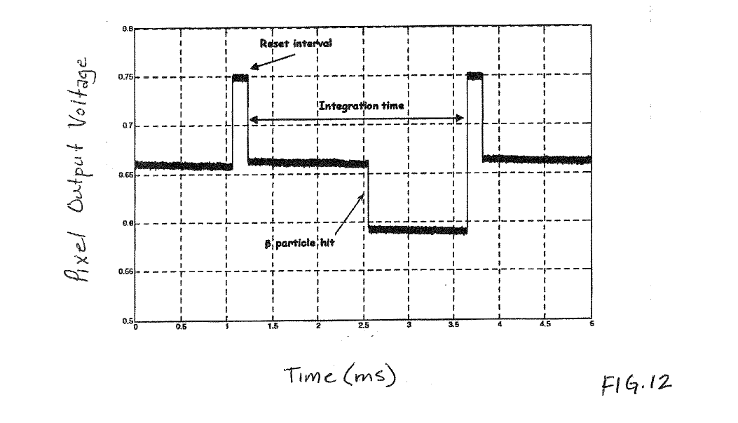

FIG. 11 schematically shows a mounting and driving arrangement for an applanator using an active pixel sensor system. Applanator 130 is mounted within support arm 132 on a pivot 140 and is counterbalanced by a mass 145. This counterbalancing leaves applanator 130 in a neutral or unbiased position in which a small force can move applanator 130. A preferred way of applying such a small force is by a solenoid or other electromagnetic means such as voice coil 150 drawn from the art of audio speakers. The electromagnetic motor can move counter mass 145 on pivot 140 to press applanator 130 variably against a cornea 133 of an eye.

Microprocessor 70 preferably controls the force applied by coil 150, while a lens 146 directs light transmitted by applanator 130 onto active pixel sensor array 50. The active pixel array delivers its output signal to microprocessor 70, which also drives an LED imager 40 that gives an operator a picture of the corneal area being contacted. The counterbalanced arm supporting applanator 130, coupled to the electromagnetic force applied by coil 150, assures that the applanator 130 is pressed against the eye lightly and more smoothly than hand force can achieve.

FIG. 12 is a diagram of the voltage output and resets that occur when a typical active pixel senses illumination. This shows the on/off effect of the preferred active pixel sensors. When this is combined with an amplifier serving each pixel, the results of the transmitted illumination are sensed accurately and completely.

What is claimed is:

1. In an applanation tonometer having a flat applanator and an illuminator of a cornea to be applanated, the improvement comprising:
a sensor arranged to sense illumination passing through the applanator in an area surrounding an applanated region of the cornea;
the sensor being an array of active pixels each having an amplifier;
the active pixel array being arranged to produce voltages based on the illumination reaching the sensor; and
a microprocessor receiving the outputs of the active pixel array and computing a size of an applanated area based on an inversion of the illumination received by the sensor.

2. The applanation tonometer of claim 1 wherein the microprocessor receiving an output from the active pixel array drives a display that images the cornea to be applanated to guide an operator in centering the applanator on the cornea.

3. The applanation tonometer of claim 1 wherein a lens focuses illumination from the applanator onto the active pixel array.

4. The applanation tonometer of claim 1 wherein the active pixel array is spaced from the applanator.

5. The applanation tonometer of claim 1 wherein a solenoid controlled by the microprocessor moves a balanced arm to press the applanator against the cornea.

6. An applanation tonometer using external illumination of a cornea to be applanated and using a plane applanating window to engage the cornea, the tonometer comprising:
a sensor of illumination passing through the applanating window;
the sensor being formed of an array of active pixels, each having an amplifier;
a microprocessor receiving total voltages from the active pixel array;
the microprocessor being programmed to calculate a size of an applanated area of the cornea as an inverse of the illumination sensed; and
an output from the active pixel array drives a display of an image of the cornea to guide a user in aligning the applanator with a center of the cornea.

7. The tonometer of claim 6 wherein a lens focusing illumination on the active pixel array is spaced from the applanator.

8. The tonometer of claim 6 wherein the active pixel array is spaced from the applanator.

9. The tonometer of claim 6 wherein the applanating window is arranged on a counterbalanced arm, and the microprocessor controls a solenoid that presses the applanating window against the cornea.

10. A method of operating an applanation tonometer to determine the size of an applanated area of a cornea being examined, the method comprising:
illuminating the cornea to be applanated so that light from the cornea passes through the applanator in regions of the applanator that do not engage the cornea;
using an active pixel sensor to receive illumination from the cornea;
programming a microprocessor to determine a size of an applanated area as an inverse of the illumination reaching the sensor; and
programming the microprocessor to use voltages from the active pixel array to drive a display of an image of the cornea to guide a user in registering the applanator with the center of the cornea.

11. The method of claim 10 including using a solenoid to press the applanator against the cornea.

12. The method of claim 11 including programming the microprocessor to energize a solenoid.

13. The method of claim 10 including counterbalancing the applanator.
